(19) United States
(12) Patent Application Publication
(10) Pub. No.: US 2010/0272667 A1
(43) Pub. Date: Oct. 28, 2010

(54) Shave Preparations

(76) Inventors: Kenneth Eugene Kyte, III, Oregonia, OH (US); Timothy Woodrow Coffindaffer, Maineville, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/768,042

(22) Filed: Apr. 27, 2010

Related U.S. Application Data
(60) Provisional application No. 61/173,109, filed on Apr. 27, 2009.

(51) Int. Cl. A61K 8/18; A61Q 9/02

(52) U.S. Cl. 424/73

(57) ABSTRACT

A shave preparation comprising water; one or more lipophilic skin conditioning agents; one or more thickening agents; one or more emulsifying agents; and one or more lubricants.

### CROSS REFERENCE TO RELATED APPLICATION

This application claims the benefit of U.S. Provisional Application No. 61/173,109 filed on Apr. 27, 2009.

### BACKGROUND OF THE INVENTION

Shave preparations are known in the art. However, problems remain with these preparations. Irritation and lack of shave closeness remain problems of these preparations. Non-limiting examples of known shave preparations include WO 93/18740, GB 02236760, U.S. Pat. No. 3,072,536, and U.S. Pat. No. 4,585,650.

Improved shave preparations that address these problems are desired in the art. This invention addresses these needs.

### SUMMARY OF THE INVENTION

One aspect of the invention provides for a shave preparation comprising water; one or more lipophilic skin conditioning agents; one or more thickening agents; one or more emulsifying agents; and one or more lubricants.

Another aspect of the invention provides for a shave preparation comprising water; one or more lipophilic skin conditioning agents; one or more thickening agents; one or more emulsifying agents; and one or more lubricants, wherein the shave preparation is substantially free of a gel network.

Yet another aspect of the invention provides for a shave preparation comprising: at least 60% water; from about 2% to about 4% of one or more lipophilic skin conditioning agents; from about 0.25% to about 3% of one or more thickening agents; from about 0.25% to about 3% of one or more emulsifying agents; and from about 0.25% to about 3% of one or more lubricants; wherein the shave preparation is substantially free of a gel network.

### DETAILED DESCRIPTION OF THE INVENTION

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

The current invention provides for shave preparations having desirable properties that improve the shave experience. Without wishing to be bound by theory, it is believed that the compositions within the shave care preparations of the present invention interact synergistically to provide for an enhanced experience.

### Water

The shave preparations of the current invention comprise water. In one embodiment, the shave preparation comprises at least about 60% by weight water. In an alternate embodiment, the shave preparation comprises at least about 70% by weight water. In an alternate embodiment, the shave preparation comprises at least about 80% by weight water. In an alternate embodiment, the shave preparation comprises at least about 90% by weight water.

### Lipophilic Skin Conditioning Agent

Shave preparations of the present invention employ one or more lipophilic skin conditioning agents. The concentration level of the skin conditioning agents either singularly or collectively may range from about 1% to about 12% by weight of the base composition. Some preferred concentration levels include greater than about 1%, from about 2% to about 5%, and from about 2% to about 4%. It is to be understood that the scope of appended claims that do not specify a concentration level of the lipophilic skin conditioning agent is not limited to the levels described in this paragraph.

Exemplary skin conditioning agents include hydrocarbons, polymeric hydrocarbons, esters, ethers, and silicones selected from the group consisting of alkyl ethers, mineral oil, isoparaffin, greater than C20 hydrogenated polyisobutene, and petrolatum; and an ester composed of a branched C16-C22 alkyl chain and a mono alkyl group consisting of a linear or branched C1 to C6 alkyl chain. Some preferred skin conditioning agents comprise isostearic acid derivatives; for example, isostearyl isostearate, isopropyl isostearate, and mixtures thereof, PPG-15 Stearyl Ether and dimethicone. Other skin conditioning agents known to the skilled artisan may also be employed depending on the form of the personal care composition and the targeted skin benefit.

The skin conditioning agents may also help to reduce the coefficient of friction for personal care compositions provided herein that are in the form of shaving compositions. The reduction in friction can decrease the potential for skin irritation that can arise from contacting the skin one or more times with a razor blade. Employment of the skin conditioning agent in this context may also permit formulation flexibility regarding the type and concentration level of lubricants (as discussed more fully below) that are included in the shaving preparations.

In one embodiment, the shave preparations of the present invention is free or substantially free of any cationic skin conditioning agents, including but not limited to cationic polymers and cationic ammonium salts. Examples of such skin conditioning agents include substituted quaternary ammonium compounds (I.e., quaterniums, stearalkonium chloride, and guar hydroxypropytrimonium chloride.) See U.S. Pat. No. 4,586,650 at col. 2 line 54 et seq for additional examples of cationic skin conditioning agents which are preferably limited or not used in the present invention. Importantly, the present invention is able to achieve a desirable shave experience without requiring said cationic skin conditioning agents. This has been found to provide desired shave performance while reducing formulation costs and complexity. As defined herein, “substantially free” means that no amount of said ingredients intentionally added into the composition, but allowing for trace amounts which may be carried over from other ingredients or from processing conditions.

### Thickening Agent (Including Thickeners and Gelling Agents)

The shave preparations of the present invention contain one or more thickening agents, from about 0.1% to about 5%, alternatively from about 0.1% to about 4%, alternatively from about 0.25% to about 3%, by weight of the composition.

Nonlimiting classes of thickening agents include those selected from the following: Carboxylic Acid Polymers, Crosslinked Polyacrylate Polymers Polyacrylamide Polymers, Polysaccharides, Clays and Gums, and mixtures thereof when appropriate.

In one embodiment, compositions of the present invention include a thickening agent selected from carboxylic acid polymers, crosslinked polyacrylate polymers, polyacrylamide polymers, polysaccharides, and mixtures thereof, more preferably selected from carboxylic acid polymers, polyacrylamide polymers, polysaccharides, and mixtures thereof.

### Emulsifier

The shave preparations of the present invention contain one or more emulsifying agents, from about 0.1% to about 8%, alternatively from about 0.1% to about 5%, alternatively from about 0.25% to about 3%, by weight of the composition.

Nonlimiting examples of surfactants for emulsification for use in the compositions of the present invention are disclosed in McCutcheon's, Detergents and Emulsifiers, North American edition (1986), published by allured Publishing Corporation; and McCutcheon's, Functional Materials, North American Edition (1992). Preferred emulsifiers are nonionic surfactants/emulsifiers. Nonlimiting useful emulsifiers herein include those selected from the group consisting of alkyl glucosides, alkyl polyglucosides, polyhydroxy fatty acid amides, alkoxylated fatty acid esters, sucrose esters, alkoxylated fatty alcohols, amine oxides, and mixtures thereof.

### Lubricants

Shave compositions of the present invention may employ one or more lubricants, from about 0.1% to about 8%, alternatively from about 0.1% to about 5%, alternatively from about 0.25% to about 3%, by weight of the composition.

Exemplary lubricants include lubricous water soluble polymers, water insoluble particles, and hydrogel-forming (or water swellable) polymers, and mixtures thereof.

Useful lubricious water soluble polymers may have a molecular weight greater between about 300,000 and 15,000,000 daltons, preferably more than about one million Daltons. Nonlimiting examples of suitable lubricious water soluble polymers include polyethylene oxide, polyvinylpyrrolidone, and polyacrylamide. Nonlimiting useful water insoluble particles may include inorganic particles or organic polymer particles. Hydrogel-forming polymers are typically highly hydrophilic polymers that, in water, form organized three-dimensional domains of approximately nanometer scale. Additional polymer lubricants include: cellulose derivatives such hydroxyalkyl cellulose polymers such as hydroxyethyl cellulose and hydroxypropyl cellulose, carboxymethyl cellulose, and cellulose methyl ether and polysaccharide gums such as, for example, xanthan gum, carrageenan gum, guar gum, locust bean gum, and hydroxypropyl guar gum.

### Gel Network

In one embodiment, the shave composition is substantially free from a gel network. As used herein, the term “gel network” refers to a lamellar or vesicular solid crystalline phase which comprises at least one fatty amphiphiles. In one embodiment, the present invention contains less than about 5%, alternatively less than about 3%, alternatively less than about 1%, alternatively less than about 0.5% of at least one fatty amphiphiles. Gel networks have been found to reduce the rinse profile of these systems. Fatty alcohol gel networks have been used for years in cosmetic creams and hair conditioners. Gel networks are a re-solidified liquid crystal gel phase formed by fatty amphiphiles (e.g. cetyl or stearyl alcohol) and a hydrophilic phase (e.g. water). It is formed by undergoing a melting and then re-solidification process in the hydrophilic phase. The gel network will typically have a lower thermal transition than the melt temperature of the fatty amphiphile itself.

### Optional Ingredients

The shave preparations can further comprise additional optional ingredients. Suitable additional optional ingredients include perfume, preservatives, chelants, sensates (e.g. menthol), desquamation actives, anti-acne actives, anti-wrinkle/anti-atrophy actives, anti-oxidants/radical scavengers, flavonoids, anti-inflammatory agents, anti-cellulite agents, topical anesthetics, tanning actives, skin lightening agents, skin soothing and healing actives, antimicrobial actives, sunscreen actives, visual skin enhancers, humectants and moisturizing agents (e.g., glycerin, glycols, sorbitol) and the like. Such optional ingredients are described more fully in U.S. application Ser. No. 11/367,918, filed Mar. 3, 2006. Preferred additional optional ingredients include salicylic acid, opacifiers (e.g. mica and titanium dioxide), perfume, hydrophilic conditioning agents (e.g., glycerin) and skin sensates (e.g. menthol).

The shave preparations of the present invention may contain salicylic acid, its isomers, tautomers, salts and derivatives thereof. Alternatively, the compositions comprise from about 0.001% to about 5% salicylic acid. Alternatively, the compositions comprise from about 0.01% to about 2% salicylic acid. Alternatively, the compositions comprise from about 0.1% to about 1% salicylic acid. Without wishing to be bound by theory, it is believed that salicylic acid is efficacious for the treatment of acne on the skin. Moreover, the salicylic acid is capable of treating and/or reducing the presence of acne on the skin. Such treatment with the shave preparation of this invention involves applying the shave preparation to the skin and shaving the skin that has been treated with the shave preparation.

Dermatologically acceptable salts include alkali metal salts, such as sodium and potassium; alkaline earth metal salts, such as calcium and magnesium; non-toxic heavy metal salts; ammonium and trialkylammonium salts such as trimethylammonium and triethylammonium. Derivatives of salicylic acid include, but are not limited to, any compounds wherein the CH3 groups are individually or in combination replaced by amides, esters, amino groups, alkyls, and alcohol esters. Tautomers of salicylic acid are the isomers of salicylic acid which can change into one another with ease so that they ordinarily exist in equilibrium. Thus, tautomers of salicylic acid can be described as having the chemical formula C7H₆O3 and generally having a similar structure to salicylic acid.

The compositions of the present invention may include from about 0.001% to about 5%, alternatively from about 0.01% to about 2%, and alternatively from about 0.1% to about 1%, of alpha- or beta-hydroxy acids, and derivatives, salts, isomers and tautomers thereof. Non-limiting examples of alpha- and beta-hydroxy acids include alpha-hydroxy-butyric acid, alpha-hydroxyisobutyric acid, alpha-hydroxyisocaproic acid, alpha-hydroxyisovaleric, atrolactic acid, beta-hydroxybutyric acid, beta-phenyl lactic acid, beta-phenylpyruvic acid, citric acid ethyl pyruvate, galacturonic acid, glucoheptonic acid, glucoheptono 1,4-lactone, gluconic acid, gluconolactone glucuronic acid, glucuronolactone, glycolic acid, isopropyl pyruvate, lactic acid, malic acid, amndelic acid, emthyl pyruvate, mucic acid, pyruvic acid, saccharic acid, saccharic acid 1,4-lactone, tartaric acid and tartronic acid, and mixtures thereof.

For shaving preparations that do not produce enough visual contrast between areas of the skin that have and have not been shaved, opacifiers may be added to the shaving preparation. Opacifiers may be either inorganic or organic compounds. Inorganic opacifiers include, for example, titanium dioxide, zinc oxide, talc, mica or coated mica (with oxides of titanium, tin, or iron or bismuth oxychloride), magnesium aluminum silicate, bismuth oxychloride, or other minerals. These compounds can be added as powders, dispersions, or complexes. Organic opacifiers include, for example, opaque emulsions (e.g., containing Styrene/PVP copolymer, vinyl polymers, or latexes), metal salts of amines containing 14-20 carbon atoms per molecule, alkanolamides containing 14-20 carbon atoms per molecule, organic alcohols containing 14-20 carbon atoms per molecule, insoluble salts of stearic acid, glycol mono-or distearates, propylene glycol and glycerol monostearates and palmitates. Combinations of these opacifiers can also be used. The opacifying additive is typically included in an amount of about 1 to about 6%, preferably about 2 to about 5%, by weight of the composition.

In one embodiment, the present composition comprises less than about 5% of one or more lathering surfactants, or less than about 3%, or less than about 2%, or less than about 1.5%, or less than about 1%, or less than about 0.5%. In another embodiment, the present composition is free or substantially free of lathering surfactants. A lathering surfactant is defined herein as surfactant, which when combined with water and mechanically agitated generates a foam or lather. Lathering surfactants include those selected from the group consisting of anionic lathering surfactants, amphoteric lathering surfactants, and mixtures thereof. Generally, the lathering surfactants are fairly water soluble. Examples of anionic lathering surfactants are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. A wide variety of anionic lathering surfactants are useful herein. Non-limiting examples of anionic lathering surfactants include those selected from the group consisting of sarcosinates, sulfates, sulfonates, isethionates, taurates, phosphates, lactylates, glutamates, and mixtures thereof. Other anionic materials useful herein are soaps (i.e., alkali metal salts, e.g., sodium or potassium salts) of fatty acids, typically having from about 8 to about 24 carbon atoms, preferably from about 10 to about 20 carbon atoms, monoalkyl, dialkyl, and trialkylphosphate salts, alkanoyl sarcosinates. Examples of zwitterionic or amphoteric surfactants are described in U.S. Pat. Nos. 5,104,646 and 5,106,609.

### EXAMPLES

The following examples are provided to further illustrate exemplary shave preparations of the present invention. Material percentages are added neat. Activity levels are specified as needed.

TABLE 1*

| Ingredient | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|
| Water | 92.25 | 92.95 | 92.05 | 92.25 | 92.95 | 91.75 | 84.30 | 88.60 | 86.90 | 84.75 |
| Sepigel 305 | 2.00 | 1.60 | 2.00 | 2.00 | 1.60 | 2.00 | 2.00 | 2.00 | 2.00 | 2.00 |
| (Polyacrylamide | | | | | | | | | | |
| & C13-14 | | | | | | | | | | |
| Isoparaffin | | | | | | | | | | |
| & Laureth-7) | | | | | | | | | | |
| Polyox | 0.50 | 0.50 | 0.50 | 0.50 | 0.50 | 0.50 | 0.50 | 0.50 | 0.40 | 0.50 |
| N12K (PEG- | | | | | | | | | | |
| 23M) | | | | | | | | | | |
| Natrosol 250 | 0.80 | 0.50 | 0.80 | 0.80 | 0.50 | 0.80 | 0.80 | 0.90 | 0.80 | 0.80 |
| HHR (HEC) | | | | | | | | | | |
| Glycerin | — | — | — | — | — | — | 5.00 | — | — | 5.00 |
| Brij 35 | 2.00 | 2.00 | 2.00 | 2.00 | 2.00 | 2.00 | 2.00 | 2.00 | 2.00 | 2.00 |
| (Laureth-23, | | | | | | | | | | |
| 100% | | | | | | | | | | |
| Active) | | | | | | | | | | |
| Salicylic | — | — | — | — | — | 0.50 | 0.50 | 0.50 | 0.50 | — |
| Acid | | | | | | | | | | |
| Arlamol E | 2.00 | 2.00 | 2.00 | — | — | 2.00 | 2.00 | 2.00 | 2.00 | 2.00 |
| (PPG-15 | | | | | | | | | | |
| Stearyl | | | | | | | | | | |
| Ether) | | | | | | | | | | |
| Niacinamide | — | — | — | — | — | — | — | — | — | 2.50 |
| Acusol | — | — | — | — | — | — | 2.50 | 2.50 | 2.50 | — |
| OP301 | | | | | | | | | | |
| Opacifier | | | | | | | | | | |
| (40% solids) | | | | | | | | | | |
| (Water and | | | | | | | | | | |
| Styrene/ | | | | | | | | | | |
| Acrylic | | | | | | | | | | |
| Copolymer) | | | | | | | | | | |
| DC 1503 | — | — | — | 2.00 | 2.00 | — | — | — | — | — |
| Expancel | — | — | — | — | — | — | — | — | 2.00 | — |
| 920-WE40 | | | | | | | | | | |
| D24 | | | | | | | | | | |
| Disodium | 0.10 | 0.10 | 0.30 | 0.10 | 0.10 | 0.10 | 0.05 | 0.10 | — | 0.10 |
| EDTA | | | | | | | | | | |
| (EDETA | | | | | | | | | | |
| BD) | | | | | | | | | | |
| Perfume 1 | 0.15 | 0.15 | 0.15 | 0.15 | 0.15 | — | — | — | — | 0.15 |
| Perfume 2 | — | — | — | — | — | — | — | — | — | — |
| Perfume 3 | — | — | — | — | — | 0.15 | 0.15 | — | — | — |
| Perfume 4 | — | — | — | — | — | — | — | 0.50 | 0.50 | — |
| Glydant Plus | 0.20 | 0.20 | 0.20 | 0.20 | 0.20 | 0.20 | 0.20 | 0.40 | 0.40 | 0.20 |

| Ingredient | Ex. 11 | Ex. 12 | Ex. 13 | Ex. 14 | Ex. 15 | Ex. 16 |
|---|---|---|---|---|---|---|
| Water | 85.24 | 84.89 | 83.3 | 84.89 | 82.83 | 80.735 |
| Cetyl Alcohol | — | — | — | 3 | 3 | 3 |
| Sepigel 305 | 2 | 2 | 2 | 1 | 1 | 1 |
| (Polyacrylamide & | | | | | | |
| C13-14 Isoparaffin & | | | | | | |
| Laureth-7) | | | | | | |
| Polyox N12K (PEG- | 0.6 | 0.8 | 0.8 | 0.8 | 0.8 | 0.8 |
| 23M) | | | | | | |
| Natrosol 250 HHR | 0.8 | 0.8 | 0.8 | 0.8 | 0.8 | 0.8 |
| (HEC) | | | | | | |
| Glycerin | — | — | — | — | 2 | — |
| Brij 35 (Laureth-23, | — | — | — | — | — | — |
| 100% Active) | | | | | | |
| Plantaren 1200 N UP | 4 | 4 | 8 | 4 | 4 | 8 |
| (Lauryl Glucoside, | | | | | | |
| 50% Active) | | | | | | |
| Lenocare SMS-30 | — | — | — | — | — | — |
| (Sodium Myristoyl | | | | | | |
| Sarcosinate, 30% | | | | | | |
| Active) | | | | | | |
| Mackam DAB-ULS | — | — | — | — | — | — |
| (Lauramidopropyl | | | | | | |
| Betaine, 35% Active) | | | | | | |
| Sodium Lauryl Sulfate | — | — | — | 0.5 | 0.5 | 0.5 |
| (29% Active) | | | | | | |
| Salicylic Acid | — | — | — | — | — | — |
| Arlamol E (PPG-15 | 2 | 2 | 2 | 2 | 2 | 2 |
| Stearyl Ether) | | | | | | |
| Petrolatum | — | — | — | — | — | — |
| Niacinamide | — | — | — | — | — | — |
| Acusol OP301 | 2.5 | 2.5 | — | — | — | — |
| Opacifier (40% solids) | | | | | | |
| (Water and Styrene/ | | | | | | |
| Acrylic Copolymer) | | | | | | |
| DC 1503 | | | | | | — |
| Expancel 920-WE40 | 2 | 2 | 2 | 2 | 2 | 2 |
| D24 | | | | | | |
| Disodium EDTA | — | — | — | — | — | — |
| (EDETA BD) | | | | | | |
| Citric Acid | 0.06 | 0.06 | 0.15 | 0.06 | 0.12 | 0.2 |
| Menthol | — | 0.05 | 0.05 | 0.05 | 0.05 | 0.065 |
| Perfume 1 | — | — | — | — | — | — |
| Perfume 2 | — | — | — | — | — | — |
| Perfume 3 | — | — | — | — | — | — |
| Perfume 4 | 0.5 | 0.6 | 0.6 | 0.6 | 0.6 | 0.6 |
| Glydant Plus | 0.3 | 0.3 | 0.3 | 0.3 | 0.3 | 0.3 |

| Ingredient | Ex. 17 | Ex. 18 | Ex. 19 |
|---|---|---|---|
| Water | 78.9 | 79.77 | 73.77 |
| Cetyl Alcohol | 3 | 3 | 3 |
| Sepigel 305 | 1 | 2 | 2 |
| (Polyacrylamide & | | | |
| C13-14 Isoparaffin & | | | |
| Laureth-7) | | | |
| Polyox N12K (PEG- | 0.8 | 0.8 | 0.8 |
| 23M) | | | |
| Natrosol 250 HHR | 0.8 | 0.8 | 0.8 |
| (HEC) | | | |
| Glycerin | 2 | — | — |
| Brij 35 (Laureth-23, | — | — | — |
| 100% Active) | | | |
| Plantaren 1200 N UP | 8 | 1 | 1 |
| (Lauryl Glucoside, | | | |
| 50% Active) | | | |
| Lenocare SMS-30 | — | 7 | 7 |
| (Sodium Myristoyl | | | |
| Sarcosinate, 30% | | | |
| Active) | | | |
| Mackam DAB-ULS | — | — | 6 |
| (Lauramidopropyl | | | |
| Betaine, 35% Active) | | | |
| Sodium Lauryl | 0.5 | 0.5 | 0.5 |
| Sulfate (29% Active) | | | |
| Salicylic Acid | — | — | — |
| Arlamol E (PPG-15 | | 2 | 2 |
| Stearyl Ether) | | | |
| Petrolatum | 2 | — | — |
| Niacinamide | — | — | — |
| Acusol OP301 | — | — | — |
| Opacifier (40% | | | |
| solids) (Water and | | | |
| Styrene/Acrylic | | | |
| Copolymer) | | | |
| DC 1503 | — | — | — |
| Expancel 920-WE40 | 2 | 2 | 2 |
| D24 | | | |
| Disodium EDTA | — | — | — |
| (EDETA BD) | | | |
| Citric Acid | 0.2 | 0.18 | 0.18 |
| Menthol | — | 0.05 | 0.05 |
| Perfume 1 | — | — | — |
| Perfume 2 | — | — | — |
| Perfume 3 | — | — | — |
| Perfume 4 | 0.5 | 0.6 | 0.6 |
| Glydant Plus | 0.3 | 0.3 | 0.3 |

*Percentages in Table 1 (Examples 1-19) are on a weight by weight basis.

The above Examples are made according to the method below.
1. Weigh out the water in a vessel sufficient to hold the entire batch
2. Insert an overhead mixer with impeller into the vessel and increase agitation to create a vortex
3. Pre-blend the thickener and polymer powders
4. Sprinkle the polymer blend into the vortex until incorporated
5. Begin heating batch to 70 C to hydrate the polymers
6. Once the batch is at 70 C, add the oil and mix until uniform and dispersed
7. Add the liquid dispersion polymer to the batch and mix until uniform and hydrated, increasing rpms to maintain good mixing
8. Add the surfactant and mix until uniform and dispersed
9. Begin cooling batch to below 45 C
10. Once below 45 C, add the perfume, preservatives and other temperature-sensitive additives
11. Cool to below 35 C and QS with water

For product with acne control actives, weigh out the solubilizing agent and salicylic acid in a separate vessel and mix until dissolved. Add to the batch during STEP 6 addition.

For product with water-soluble actives, add to the batch during STEP 10 additions

For product with oil-soluble actives, add to the batch during STEP 6 additions

For product with improved tracking, add the opacifier during the STEP 10 additions

For product with lower density, add the Expancel to the batch during STEP 6 additions

While the specification concludes with the claims particularly pointing and distinctly claiming the invention, it is believed that the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All percentages and ratios used herein are by weight of the total composition and all measurements made are at 25° C., unless otherwise designated. A degree is a planar unit of angular measure equal in magnitude to 1/360 of a complete revolution.

All measurements used herein are in metric units unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A shave preparation comprising:
water;
one or more lipophilic skin conditioning agents;
one or more thickening agents;
one or more emulsifying agents; and
one or more lubricants.

2. The shave preparation of claim 1, wherein the shave preparation comprises from about 1% to about 12% by weight of the one or more lipophilic skin conditioning agents.

3. The shave preparation of claim 1, wherein the shave preparation comprises from about 0.1% to about 5% by weight of the one or more thickening agents.

4. The shave preparation of claim 1, wherein the shave preparation comprises from about 0.1% to about 8% by weight of the one or more emulsifying agents.

5. The shave preparation of claim 1, wherein the shave preparation comprises from about 0.1% to about 8% by weight of the one or more lubricants.

6. The shave preparation of claim 1, further comprising one or more of a salicylic acid, an opacifier, and a combination thereof.

7. The shave preparation of claim 6, wherein the shave preparation comprises from about 0.001% to about 5% by weight of the salicylic acid.

8. The shave preparation of claim 6, wherein the shave preparation comprises from about 1 to about 6%, by weight of the opacifier.

9. The shave preparation of claim 1, substantially free of a cationic skin conditioning agent.

10. The shave preparation of claim 1, comprising less than 2% of a lathering surfactant.

11. A shave preparation comprising:
water;
one or more lipophilic skin conditioning agents;
one or more thickening agents;
one or more emulsifying agents; and
one or more lubricants;
wherein the shave preparation is substantially free of a gel network.

12. The shave preparation of claim 10, wherein the shave preparation comprises from about 1% to about 12% by weight of the one or more lipophilic skin conditioning agents.

13. The shave preparation of claim 10, wherein the shave preparation comprises from about 0.1% to about 5% by weight of the one or more thickening agents.

14. The shave preparation of claim 10, wherein the shave preparation comprises from about 0.1% to about 8% by weight of the one or more emulsifying agents.

15. The shave preparation of claim 10, wherein the shave preparation comprises from about 0.1% to about 8% by weight of the one or more lubricants.

16. The shave preparation of claim 10, further comprising salicylic acid, an opacifier, or a mixture thereof.

17. The shave preparation of claim 16, wherein the shave preparation comprises from about 0.001% to about 5% by weight of the salicylic acid.

18. The shave preparation of claim 16, wherein the shave preparation comprises from about 1 to about 6%, by weight of the opacifier.

19. A shave preparation comprising:
at least 60% water;
from about 2% to about 4% of one or more lipophilic skin conditioning agents;
from about 0.25% to about 3% of one or more thickening agents;
from about 0.25% to about 3% of one or more emulsifying agents; and
from about 0.25% to about 3% of one or more lubricants;
wherein the shave preparation is substantially free of a gel network.

20. A method of treating acne on skin comprising applying the shave preparation of claim 6 to the skin and shaving the skin.
